(19) United States
(12) Patent Application Publication
(10) Pub. No.: US 2020/0046855 A1
(43) Pub. Date: Feb. 13, 2020

(54) CONTRAST MIXTURE AND USE THEREOF

(71) Applicants: Andrea JAKABCINOVÁ, Kosice (SK); Csilla ULICNÁ, Presov (SK); UNIVERZITA PAVLA JOZEFA SAFÁRIKA V KOSICIACH, Kosice (SK)

(72) Inventors: Andrea JAKABCINOVÁ, Kosice (SK); Csilla ULICNÁ, Presov (SK); Marta PÚCATOVÁ, Liptovská Stiavnica (SK); Patrik JAKABCIN, Kosice (SK); Jozef ULICNÝ, Presov (SK)

(73) Assignee: Univerzita Pavla Jozefa Safárika V, Kosice (SK)

(21) Appl. No.: 16/339,446

(22) PCT Filed: Oct. 5, 2017
(86) PCT No.: PCT/IB2017/056150

§ 371 Date: Apr. 4, 2019

(30) Foreign Application Priority Data
Oct. 6, 2016 (SK) PP 50064-2016
Oct. 6, 2016 (SK) PUV 50107-2016

(51) Int. Cl. A61K 49/00

(52) U.S. Cl. CPC A61K 49/006; A61K 49/0076

(57) ABSTRACT

A contrast mixture with increased affinity and selectivity to neoplasms of the gastrointestinal tract, being of three components and consisting of 0.0005-0.01% by weight of low-molecular component, 0.05-6% by weight of high-molecular component and isotonic solution, wherein low-molecular component is a contrast substance, which is a colouring agent used in food industry and pharmacy and high-molecular component is a colloid modulator of velocity designed to decelerate diffusion of the contrast mixture into a tissue in such manner that the velocity of diffusion into healthy tissue of gastrointestinal tract and the velocity of diffusion into neoplasm tissue of gastrointestinal tract is different. In an embodiment, the colouring agent is the sodium and/or calcium salt of [4-(alpha-(4-diethylaminophenyl)-5-hydroxy-2,4-disulphophenylmethylidene)-2,5-cyclohexadiene-1-ylidene] diethylammonium hydroxide or 3,7-bis(Dimethylamino)-phenothia zin-5-ium chloride and the colloid modulator of velocity is a starch, pectin, agar-agar (agarose and agaropectins) and/or non-linearly branched amylopectins. A use of the contrast mixture for colour sharp distinction of the interface between healthy tissue and neoplasm tissue of gastrointestinal tract.

### TECHNICAL FIELD

The invention concerns contrast mixture and use thereof and belongs to the medical field.

### BACKGROUND ART

Neoplasms of gastrointestinal tract are tumours—transformed tissues. When untreated, they can turn with high probability into malignant forms. The treatment of such neoplasms consists usually of radical, i.e. surgical intervention that removes the detected neoplasm. The location, shape and size of neoplasm is in such case determined by endoscopic techniques where a surgeon by means of endoscopic optical probe localizes an affected section of gastrointestinal tract. Neoplasms which do not grow into deeper layers of tissue can be removed during the same endoscopic examination by using techniques of polypectomy, i.e. application of polypectomy loop and tissue removal (“jumbo biopsy”, “strip biopsy”) via rigid sigmoidoscope. These techniques commonly known as endoscopic mucosal resection (EMR) and endoscopic resection (ER) of the lift&cut type require submucosal injection before the actual intervention. The purpose of injecting such solution into submucosal tissue is to incite separation of the neoplasm (its elevation) from muscularis propria. The protrusion of the neoplasm enables application of the polypectomy loop and control of a cutting surface.

Composition of the injection solution for use for EMR and ER is not standardized. In literature is mentioned the use of a coagulant—a highly concentrated salt solution with addition of adrenaline or epinephrine (Conio, Massimo. “Endoscopic Mucosal Resection.” Gastroenterology & Hepatology 7, no. 4 (April 2011): 248-50. http://www.ncbi.nlm.nih.gov/pmc/articles/PMC3127027), a physiological solution with addition of methylene blue or sodium salt of indigotindisulfonate as a visual tool staining the neoplasm (Conio, Massimo. “Endoscopic Mucosal Resection.” Gastroenterology & Hepatology 7, no. 4 (April 2011): 248-50. http://www.ncbi.nlm.nih.gov/pmc/articles/PMC3127027) and also a physiological solution with addition of cellulose derivatives, succinylated gelatin, glycerol and fibrinogen solution as diffusion inhibitor (Hwang, Joo Ha, Vani Konda, Barham K. Abu Dayyeh, Shailendra S. Chauhan, Brintha K. Enestvedt, Larissa L. Fujii-Lau, Sri Komanduri, et al. “Endoscopic Mucosal Resection.” Gastrointestinal Endoscopy 82, no. 2 (August 2015): 215-26. doi:10.1016/j.gie.2015.05.001).

The main disadvantage of agents used until now for diagnostics and surgical treatment of neoplasms of the gastrointestinal tract is a short period of time of the protrusion of neoplasm (separation of neoplasm) and its visual differentiating from surrounding tissue. The time of existence of thus created pseudopolyp is determined by fast diffusion of the injected mixture, which results in the disappearance of the protrusion of neoplasm without its colour delimitation. It is this time of protrusion of neoplasm which determines a timeframe usable for intervention. In case that additional colouring agent is used, the interface between the neoplasm and healthy tissue is diffused (i.e. unbounded, dispersed) as a result of fast diffusion of the colouring agent into both volumes.

In the document US 2005/0171419 A1 an extremely widely formulated mechanism of a medium with time-dependent viscosity for use in various displaying modalities of intracorporeal cavities is disclosed. The disclosed medium is administered into intracorporeal cavities, and not by injection into muscularis propria, i.e. into tissue, and the mechanism of its action is based on the viscosity change of the administered medium.

In the document US 2008/0008656 A1, a fluorescent staining of a lumen of gastrointestinal tract by fluorescent agent is disclosed and the contrast effect between neoplasm and surrounding tissue is achieved by use of a fluorescence.

In the document EP 0 627 632 B1, a preparation of the contrast substance making use of physical properties of magnetic resonance (MM), wherein the contrast factor for MRI consists of magnetic particles bound in the carrier, is disclosed.

In the document U.S. Pat. No. 7,582,283 B2, the contrast factor intended for requirements of computer tomography (CT) in abdominal and pelvis area is disclosed. The physical principle of CT contrast is based on the contrast within X-ray region, not in the visible region.

In the document U.S. Pat. No. 5,653,959, an image contrast enhancement during diagnostic examination of gastrointestinal tract by general method of displaying in (visible) light (“light imaging”) for requirement of ultrasound echography and nuclear magnetic resonance (NMM) while using paramagnetic substances (metals in ionic or organometallic state, e.g. Fe, Gd³⁺).

In the document U.S. Pat. No. 4,986,256, the composition of the contrast substance using paramagnetic metalloporphyrins for the Mill requirements is disclosed.

In the document U.S. Pat. No. 5,370,901, a making of a contrast mixture for ultrasonic imaging based on microparticles is disclosed.

In the document U.S. Pat. No. 6,241,968 B1, the contrast composition is disclosed, wherein a contrast factor is a variant of paramagnetic metal chelates intended for the MRI requirements.

In the document U.S. Pat. No. 5,429,814, the contrast substance which contains transition metals or rare earth metals for gastrointestinal tract contrasting in MRI studies is described. These metals are protected from a direct accession in molecular sieves.

The aim of this invention is to find a composition of injection solution for use in diagnostics and surgical treatment of neoplasms of gastrointestinal tract having increased affinitive and selective effect for diagnostics and surgical treatment of neoplasms of gastrointestinal tract, which improves a visual control of neoplasm and healthy tissue during intervention, which prolongs timeframe of the intervention and thereby allows for higher precision and quality of polypectomy. Better visual feedback during the surgery enables more precise intervention even in complicated conditions (e.g. limited access for polypectomy loop or possible proximity to an inoperable tumour).

### NATURE OF INVENTION

A contrast mixture with increased affinitive and selective effect on neoplasms of gastrointestinal tract according to this invention, the nature of which lies in that it is of three-component and consists of 0.0005%-0.01% by weight of low-molecular component and 0.05%-6% by weight of high-molecular component and isotonic solution. Low-molecular component is a contrast substance and high-molecular component is a colloid modulator of velocity designed to decelerate diffusion of the contrast mixture into tissue. The colloid modulator of velocity is a component which must satisfy a condition that velocity of diffusion into healthy tissue of gastrointestinal tract and velocity of diffusion into neoplasm tissue of gastrointestinal tract is different. The contrast substance is a colouring agent used in food industry and pharmacy.

As colloid modulators of velocity, polysaccharide linear polymers—starches or pectins or agar-agar (agarose and agaropectins) or non-linearly branched amylopectins or mixtures thereof—can be used.

The size of the colloid particles acting as diffusion velocity modulators is within the range of 1-1000 nm. Content of the contrast substance within the scope of this invention can be adjusted to the morphological type of gastrointestinal finding to obtain optimal visual contrast.

The colloid modulator of velocity can be a macromolecular substance that ensures passage through bioptic channel during examination, but at the same time decelerates diffusion to prolong duration of elevation of polyps.

Ratio of agarose and agaropectins in the contrast mixtures must be such that passage of the contrast mixture through the bioptic channel during examination is ensured, but at the same time diffusion is decelerated to prolong duration of elevation of polyps. Agarose and agaropectin are native components of agar-agar, a natural compound, which can be synthesised, but natural source is used (isolation from sea algae) and modification of components ratio is made by precipitation of agaropectin.

Agarose is a complex of polysaccharide chains composed of alternating units of α-(1-3)-D-galactosyl-β-(1-4)-anhydro-L-galactosyl.

A polymer of α-(1-4)-linked D-galacturonic acid is an example of a linear pectin usable as modulator of velocity.

As modulator of velocity in the contrast mixture according to this invention, following components can be used, which are identified by theirs CAS Registry Number:

Agar: CAS Registry Number: 9002-18-0

Agarose: CAS Registry Number: 9012-36-6

Agaropectin: v CAS only as a part of Agar.

Pectin: beta-pectin: CAS Registry Number: 9000-69-5

Amylopectin: CAS Registry Number: 9037-22-3

Low-molecular component can be sodium and/or calcium salt of [4-(α-(4-diethylaminophenyl)-5-hydroxy-2,4-disulphophenylmethylidene)-2,5-cyclohexadiene-1-ylidene] diethylammonium hydroxide and high-molecular component can be hydroxymethyl starch and/or hydroxyethyl starch.

The contrast mixture according to this invention is used to achieve colour sharp distinction of interface between healthy tissue and neoplasm tissue of the gastrointestinal tract as well as simultaneously enlargement of volume of neoplasm of gastrointestinal tract and thus its protrusion from healthy tissue of the gastrointestinal tract, in duration of 10-25 minutes in the visible region. Unlike the preparations used until now, in which because of the fast diffusion of the colouring agent into both volumes, the interface of neoplasm tissue is diffuse (unbounded, dispersed), when the contrast mixture according to the invention is used, the interface between healthy tissue of gastrointestinal tract and neoplasm tissue is sharply distinct in colour. The sharp distinct colour interface is achieved by different velocity of diffusion of the low-molecular component and the high-molecular component into tissue. The neoplasm forms a structure in which epithelial cells are interconnected, thus the macromolecular components permeate slower from the lumen of gastrointestinal tract into the wall of neoplasm than low-molecular components.

When using the contrast mixture according to this invention, the prolongation of the time period of neoplasm tissue being in contrast with healthy tissue is achieved, in duration of 10-25 minutes and the contrasting is negative.

The interface between healthy and neoplasm tissue is a different tissue as it contains elements of both tissues (cells and extracellular matrix) and high-molecular component permeates into tissue of interface with different velocity than into healthy tissue and neoplasm tissue, and thus a new colouring of this thin interface is achieved.

When using the contrast mixture according to this invention, the prolongation of a timeframe of intervention to the period of 10-25 minutes is achieved. At the same time, the colour sharp distinction of interface between healthy tissue and neoplasm, as well as the volume of the protruded neoplasm itself, increase a precision and quality of polypectomy. The better visual feedback during surgery enables more precise intervention even in complicated conditions (e.g. poor accessibility for polypectomy loop or proximity of inoperable tumour).

This prolongation of an applicability period reduces time stress of the medical staff and comforts the patient (the intervention falls within an outpatient care, it is performed under local anaesthesia and patient is fully conscious). Improved visual information during endoscopic diagnostics and intervention itself decreases invasiveness of the intervention (less of the healthy tissue being removed), reduces cognitive load for medical staff, shortens the duration of intervention, minimizes risks for the patient (perforation of intestine and/or duodenum wall) and improves surgeon's comfort during application of ligator and subsequent resection. The visual contrast enables to localize neoplasm more precisely and thus reduces resection of healthy tissue.

The contrast mixture according to this invention enables drop-in replacement (i.e. direct replacement of an existing agent by a new agent without requirement for change of protocol and instrumentation) of existing contrast substances with prolongation of time interval available for surgical intervention and improvement of its precision without increase in unit cost.

### OVERVIEW OF FIGURES

FIG. 1—Endoscopic finding before submucosal injection of contrast mixture.

FIG. 2—Endoscopic finding after submucosal injection—injection site.

### EXAMPLES OF EMBODIMENTS

The proposed invention is further explained by means of following non-restrictive examples. Even though a number of embodiments is further disclosed, it is obvious that a skilled person in the art will find other possible alternatives to these embodiments. Therefore the scope of the invention is not restricted to these embodiments disclosed in examples but is given by definition of patent claims.

### Example 1

The prepared contrast mixture for diagnostics and surgical treatment of neoplasms of gastrointestinal tract according to this invention has following composition:

Low-molecular component, i.e. the contrast substance, which is in this example of embodiment the sodium salt of [4-(α-(4-diethylaminophenyl)-5-hydroxy-2,4-disulphophenylmethylidene)-2,5-cyclohexadiene-1-ylidene] diethylammonium hydroxide in amount: 0.025 g, i.e. 0.0025% by weight.

High-molecular component, i.e. the colloid modulator of velocity, which is in this example hydroxyethyl starch in amount: 30.000 g, i.e. 3.0000% by weight.

Isotonic solution in amount: 969.975 g, i.e. 96.9975% by weight.

The content of the contrast substance in the range according to this invention can be adjusted to a morphologic type of finding in gastrointestinal tract so that the optimal visual contrast is obtained.

The colloid modulator of velocity provides deceleration of diffusion of the contrast mixture into tissue, wherein the velocity of diffusion into healthy tissue and the velocity of diffusion into neoplasm tissue of the gastrointestinal tract is different.

The contrast mixture according to this invention was used for diagnostics and surgical treatment of the neoplasm of large intestine.

By using the contrast mixture according to this invention for diagnostics and surgical treatment of neoplasms of gastrointestinal tract, the colour sharp distinction of interface between healthy tissue and neoplasm tissue of gastrointestinal tract was achieved in the visible area as well as increase in volume of the neoplasm of gastrointestinal tract and thus its protrusion from healthy tissue of gastrointestinal tract, namely in duration of typical 15 minutes. The colour sharp distinction of interface is achieved by a different velocity of diffusion of low-molecular component and high-molecular component diffusion into healthy tissue and neoplasm tissue.

The neoplasm of gastrointestinal tract forms a structure whose epithelial cells are interconnected, so macromolecular components permeate from lumen of gastrointestinal tract into the wall of neoplasm slower than low-molecular components.

Differences in permeating of high-molecular component of the mixture into tissue are quantitative, not qualitative, i.e. high-molecular component permeates into healthy tissue with different velocity than into neoplasm. Injection of the contrast substance is applied into healthy tissue (injected under neoplasm), thus initial diffusion of the contrast mixture occurs in the administration site (injection site) of healthy tissue. Diffusion is a dynamic process that results in an increased concentration of colouring agent in neoplasm tissue compared to the healthy one during therapeutic time window, i.e. colour distinction of neoplasm tissue from healthy tissue is achieved (dark blue-light blue) and contrasting is negative. Colour transition between healthy tissue and neoplasm tissue is formed by a different tissue which comprises elements of both tissues (cells and extracellular matrix) whereby a new turquoise coloured staining of this narrow interface is achieved.

By using the contrast mixture according to this invention, prolongation of timeframe of intervention for typical 15 minutes is achieved without repeated administration of the contrast mixture. At the same time, the sharp difference in colour of interface between healthy tissue and neoplasm tissue, as well as actual volume of protruded neoplasm, increases precision and quality of polypectomy. Better visual control during surgery enables more precise intervention even under complicated conditions (e.g. worse access for polypectomy loop or possible proximity of inoperable tumour).

This prolongation of an applicability period reduces time stress of the medical staff and comforts the patient (the intervention falls within an outpatient care, it is performed under local anaesthesia and patient is fully conscious). An improved visual feedback during endoscopic diagnostics and intervention itself decreases invasiveness of the intervention (less of the healthy tissue being removed), cognitive load for medical staff, shortens the duration of intervention, minimizes risks for the patient (perforation of intestine wall and duodenum wall) and improves surgeon's comfort during application of ligator and subsequent resection. The visual contrast enables to localize neoplasm more precisely and thus reduces resection of healthy tissue.

The contrast substance according to this invention is applicable for selective contrasting of neoplasms along the whole length of gastrointestinal tract.

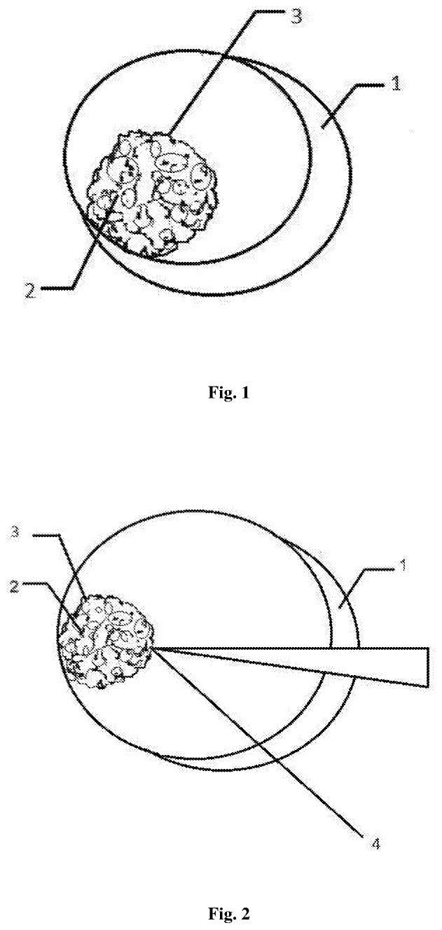

In FIGS. 1 and 2 is shown documentation that is related to the polypectomy of large intestine neoplasm when using the contrast mixture according to this example of embodiment.

### Example 2

The contrast mixture for diagnostics and surgical treatment of neoplasms of gastrointestinal tract according to this invention has similar composition as in Example of embodiment 1 with the difference that instead of the sodium salt of [4-(α-(4-diethylaminophenyl)-5-hydroxy-2,4-disulphophenylmethylidene)-2,5-cyclohexadiene-1-ylidene] diethylammonium hydroxide, the calcium salt of [4-(α-(4-diethylaminophenyl)-5-hydroxy-2,4-disulphophenylmethylidene)-2,5-cyclohexadiene-1-ylidene] diethylammonium hydroxide is used and instead of hydroxyethyl starch, the hydroxymethyl starch is used.

Other factors are the same as stated in the Example of embodiment 1.

### Example 3

The contrast mixture for diagnostics and surgical treatment of neoplasms of gastrointestinal tract according to this invention has similar composition as in Example of embodiment 1 with the difference that instead of sodium salt of [4-(α-(4-diethylaminophenyl)-5-hydroxy-2,4-disulphophenylmethylidene)-2,5-cyclohexadiene-1-ylidene] diethylammonium hydroxide, the mixture of sodium and calcium salt of [4-(α-(4-diethylaminophenyl)-5-hydroxy-2,4-disulphophenylmethylidene)-2,5-cyclohexadiene-1-ylidene] diethylammonium hydroxide is used in 1:1 ratio, wherein the mixture is in amount of 0.0025% by weight.

Instead of hydroxyethyl starch the mixture of hydroxyethyl starch and hydroxymethyl starch in 1:1 ratio is used, wherein the mixture is in amount of 3.00% by weight.

Other factors are the same as stated in Example of embodiment 1.

### Example 4

The contrast mixture for diagnostics and surgical treatment of neoplasms of gastrointestinal tract according to this invention has similar composition as in Example of embodiment 1 with the difference that instead of sodium salt of [4-(α-(4-diethylaminophenyl)-5-hydroxy-2,4-disulphophenylmethylidene)-2,5-cyclohexadiene-1-ylidene] diethylammonium hydroxide, the mixture of sodium and calcium salt of [4-(α-(4-diethylaminophenyl)-5-hydroxy-2,4-disulphophenylmethylidene)-2,5-cyclohexadiene-1-ylidene] diethylammonium hydroxide in 3:7 ratio is used, wherein the mixture is in amount of 0.0025% by weight.

Instead of hydroxyethyl starch the mixture of hydroxyethyl starch and hydroxymethyl starch in 1:1 ratio is used, wherein the mixture is in amount of 3.00% by weight.

Other factors are the same as stated in Example of embodiment 1.

### Example 5

The contrast mixture for diagnostics and surgical treatment of neoplasms of gastrointestinal tract according to this invention has similar composition as in Example of embodiment 1 with the difference that instead of sodium salt of [4-(α-(4-diethylaminophenyl)-5-hydroxy-2,4-disulphophenylmethylidene)-2,5-cyclohexadiene-1-ylidene] diethylammonium hydroxide, the mixture of sodium and calcium salt of [4-(α-(4-diethylaminophenyl)-5-hydroxy-2,4-disulphophenylmethylidene)-2,5-cyclohexadiene-1-ylidene] diethylammonium hydroxide in 7:3 ratio is used, wherein the mixture is in amount of 0.0025% by weight.

And instead of hydroxyethyl starch, the mixture of hydroxyethyl starch and hydroxymethyl starch in 1:1 ratio is used, wherein the mixture is in amount of 3.00% by weight.

Other factors are the same as stated in Example of embodiment 1.

### Example 6

The prepared contrast mixture for diagnostics and surgical treatment of neoplasms of gastrointestinal tract according to this invention has following composition:

Low-molecular component, i.e. the contrast substance, which is in this example of embodiment the sodium salt of [4-(α-(4-diethylaminophenyl)-5-hydroxy-2,4-disulphophenylmethylidene)-2,5-cyclohexadiene-1-ylidene] diethylammonium hydroxide in amount: 0.025 g, i.e. 0.0025% by weight.

High-molecular component, i.e. the colloid modulator of velocity, which is in this case the mixture of agarose and agaropectin in 7:3 ratio in total amount: 1.5000 g, i.e. 0.15000% by weight.

Isotonic solution in amount: 998.745 g, i.e. 99.8745% by weight.

Other factors are the same as stated in Example of embodiment 1.

### Example 7

The prepared contrast mixture for diagnostics and surgical treatment of neoplasms of gastrointestinal tract according to this invention has following composition:

Low-molecular component, i.e. the contrast substance, which is in this example of embodiment 3,7-bis(Dimethylamino)-phenothiazin-5-ium chloride in amount: 0.025 g, i.e. 0.0025% by weight.

High-molecular component, i.e. the colloid modulator of velocity, which is in this example of the embodiment the mixture of agarose and agaropectin in 7:3 ratio in total amount: 1.5000 g, i.e. 0.15000% by weight.

Isotonic solution in amount: 998.745 g, i.e. 99.8745% by weight.

Other factors are the same as stated in Example of embodiment 1.

### Example 8

The prepared contrast mixture for diagnostics and surgical treatment of neoplasms of gastrointestinal tract according to this invention has following composition:

Low-molecular component, i.e. the contrast substance, which is in this example of embodiment the sodium salt of [4-(α-(4-diethylaminophenyl)-5-hydroxy-2,4-disulphophenylmethylidene)-2,5-cyclohexadiene-1-ylidene] diethylammonium hydroxide in amount: 0.025 g, i.e. 0.0025% by weight.

High-molecular component, i.e. the colloid modulator of velocity, which is in this case the mixture of agarose and agaropectin in 1:1 ratio in total amount: 1.5000 g, i.e. 0.15000% by weight.

Isotonic solution in amount: 998.745 g, i.e. 99.8745% by weight.

Other factors are the same as stated in Example of embodiment 1.

### Example 9

The prepared contrast mixture for diagnostics and surgical treatment of neoplasms of gastrointestinal tract according to this invention has following composition:

Low-molecular component, i.e. the contrast substance, which is in this example of embodiment the sodium salt of [4-(α-(4-diethylaminophenyl)-5-hydroxy-2,4-disulphophenylmethylidene)-2,5-cyclohexadiene-1-ylidene] diethylammonium hydroxide in amount: 0.025 g, i.e. 0.0025% by weight.

High-molecular component, i.e. the colloid modulator of velocity, which is in this case the mixture of agarose and agaropectin in 3:7 ratio in total amount: 1.5000 g, i.e. 0.15000% by weight.

Isotonic solution in amount: 998.745 g, i.e. 99.8745% by weight.

Other factors are the same as stated in Example of embodiment 1.

### Example 10

The prepared contrast mixture for diagnostics and surgical treatment of neoplasms of gastrointestinal tract according to this invention has following composition:

Low-molecular component, i.e. the contrast substance, which is in this example of embodiment the 3,7-bis(Dimethylamino)-phenothiazin-5-ium chloride in amount: 0.025 g, i.e. 0.0025% by weight.

High-molecular component, i.e. the colloid modulator of velocity, which is in this example of the embodiment the mixture of agarose and agaropectin in 1:1 ratio in total amount: 1.5000 g, i.e. 0.15000% by weight.

Isotonic solution in amount: 998.745 g, i.e. 99.8745% by weight.

Other factors are the same as stated in Example of embodiment 1.

### Example 11

The prepared contrast mixture for diagnostics and surgical treatment of neoplasms of gastrointestinal tract according to this invention has following composition:

Low-molecular component, i.e. the contrast substance, which is in this example of embodiment the 3,7-bis(Dimethylamino)-phenothiazin-5-ium chloride in amount: 0.025 g, i.e. 0.0025% by weight.

High-molecular component, i.e. the colloid modulator of velocity, which is in this example of the embodiment the mixture of agarose and agaropectin in 3:7 ratio in total amount: 1.5000 g, i.e. 0.15000% by weight.

Isotonic solution in amount: 998.745 g, i.e. 99.8745% by weight.

Other factors are the same as stated in Example of embodiment 1.

### Example 12

The prepared contrast mixture for diagnostics and surgical treatment of neoplasms of gastrointestinal tract according to this invention has following composition:

Low-molecular component, i.e. the contrast substance, which is in this example of embodiment the 3,7-bis(Dimethylamino)-phenothiazin-5-ium chloride in amount: 0.025 g, i.e. 0.0025% by weight.

High-molecular component, i.e. the colloid modulator of velocity, which is in this example of the embodiment the amylopectin: alpha-D-gluco-hexopyranosyl-(1->4)-alpha-D-gluco-hexopyranosyl-(1->6)-[alpha-D-gluco-hexopyranosyl-(1->4)]-alpha-D-gluco-hexopyranosyl-(1->4)-alpha-D-gluco- hexopyranose in amount: 1.5000 g, i.e. 0.15000% by weight.

Isotonic solution in amount: 998.745 g, i.e. 99.8745% by weight.

Other factors are the same as stated in Example of embodiment 1.

### Example 13

The prepared contrast mixture for diagnostics and surgical treatment of neoplasms of gastrointestinal tract according to this invention has following composition:

Low-molecular component, i.e. the contrast substance, which is in this example of embodiment the sodium salt of [4-(α-(4-diethylaminophenyl)-5-hydroxy-2,4-disulphophenylmethylidene)-2,5-cyclohexadiene-1-ylidene] diethylammonium hydroxide in amount: 0.025 g, i.e. 0.0025% by weight.

High-molecular component, i.e. the colloid modulator of velocity, which is in this case the pectin: (2S, 3R, 4S, 5R, 6R)-3,4,5,6-tetrahydroxyoxane-2-carboxylic acid in total amount: 1.5000 g, i.e. 0.15000% by weight.

Isotonic solution in amount: 998.745 g, i.e. 99.8745% by weight.

Other factors are the same as stated in Example of embodiment 1.

### REFERENCE SIGNS

1—Gastrointestinal tract
2—Neoplasm of gastrointestinal tract
3—Interface between gastrointestinal tract and neoplasm of gastrointestinal tract
4—Injection site

### INDUSTRIAL APPLICABILITY

The contrast mixture according to this invention is characterized by selective contrasting of neoplasms along the whole length of gastrointestinal tract and thus it is possible to use it also for other neoplasms localised in other parts of gastrointestinal tract such as small intestine or duodenum.

1. A contrast mixture with increased affinity and selectivity to neoplasms of the gastrointestinal tract, wherein the contrast mixture comprises three components and consists of 0.0005-0.01% by weight of a low-molecular component, 0.05-6% by weight of a high-molecular component and an isotonic solution, wherein the low-molecular component is a contrast substance comprising a sodium and/or calcium salt of [4-(α-(4-diethylaminophenyl)-5-hydroxy-2,4-disulphophenylmethylidene)-2,5-cyclohexadiene-1-ylidene] diethylammonium hydroxide or 3,7-bis(Dimethylamino)-phenothiazin-5-ium chloride and the high-molecular component is a colloid modulator of velocity
which is comprising a polysaccharide linear polymer.

2. The contrast mixture according to claim 1, wherein the polysaccharide linear polymer is starch, pectin, agar-agar (agarose and agaropectins), non-linearly branched amylopectins or combinations thereof, wherein the size of colloid particles is within the range of 1-1000 nm.

3. The contrast mixture according to claim 2, wherein the starch is hydroxymethyl starch and/or hydroxyethyl starch.

4. A method of utilizing the contrast mixture according to claim 1 for colour sharp distinction of interface between healthy tissue and neoplasm tissue of gastrointestinal tract in duration of 10-25 minutes, for enlargement of volume of a protruded neoplasm of the gastrointestinal tract in duration of 10-25 minutes, or for colour contrasting of healthy tissue and neoplasm tissue in duration of 10-25 minutes without repeated administration of the contrast mixture.
